(19) United States
(12) Patent Application Publication
(10) Pub. No.: US 2021/0068360 A1
(43) Pub. Date: Mar. 11, 2021

(54) STEVIA PLANTS WITH AN INCREASED REBAUDIOSIDE D CONTENT

(71) Applicant: CARGILL, INCORPORATED, Wayzata, MN (US)

(72) Inventors: Robert Joshua BROWER, III, Clayton, OH (US); Ting Liu CARLSON, Marletta, SC (US); Benyuan DANG, Fort Collins, CO (US); Moises David GONZALEZ, Camarillo, CA (US); Molly McDonald KENNEDY, Fort Collins, CO (US); Nathan Edward KNUTSON, Maple Grove, MN (US)

(73) Assignee: CARGILL, INCORPORATED, Wayzata, MN (US)

(21) Appl. No.: 16/987,713

(22) Filed: Aug. 7, 2020

Related U.S. Application Data
(62) Division of application No. 14/774,440, filed on Sep. 10, 2015, now abandoned.
(60) Provisional application No. 61/792,796, filed on Mar. 15, 2013.

(51) Int. Cl. A01H 5/10; A01H 5/12; C07K 14/415; A23L 27/30

(52) U.S. Cl. CPC A01H 5/10; A01H 5/12; A23V 2002/00; A23L 27/36; C07K 14/415

(57) ABSTRACT

Plants, and methods of generating plants, having an increased content of rebaudioside D and oilier desirable characteristics are disclosed. Rebaudioside D (rebD) has been observed to have desirable sweetening properties, and accordingly, it has been desirable to produce Stevia plant lines, cultivars, and varieties with defined glycoside profiles where rebD is increased in content, either on a total weight basis of Stevia leaves, relative to other glycosides (e.g., rebA, or stevioside), or both.

This application claims the benefit of U.S. Provisional Application No. 61/792,796, filed on Mar. 15, 2013, the entirety of which is incorporated by reference herein.

### BACKGROUND

Stevia Rebaudiana (e.g., Stevia Rebaudiana Bertoni, hereinafter “Stevia”) is believed to have been discovered in South America in the early 1900s. The leaves of the plant are generally considered more than an order of magnitude sweeter than sugar cane due to the presence of a series of glycosides. The plant and its glycosides are considered desirable for use as a low calorie sweetener. Among the sweetening components present in the leaves of Stevia plants are stevioside, rebaudioside A, B, C, D, E, F, dulcoside A (DA), steviolbioside, rubusoside, and the like, each of which has its own particular sweetness and associated tastes. The quality of sweetness is subtle, and even purified stevioside, which is one of if not the major glycoside in Stevia plants, is believed to have an unpleasant bitter aftertaste compared to rebaudioside A. It has been observed that, like rebaudioside A (“rebA”), rebaudioside (“rebD) has desirable sweetening qualities, properties, and taste.

### SUMMARY

Rebaudioside D (rebD) has been observed to have desirable sweetening properties, and accordingly, it has been desirable to produce Stevia plant lines, cultivars, and varieties with defined glycoside profiles where rebD is increased in content, either on a total weight basis of Stevia leaves, relative to other glycosides (e.g., rebA, or stevioside), or both. Such Stevia plants not only produce more of the specifically desirable sweetening components (i.e., the Stevia glycoside profile is high in rebD), but also offer ease of processing by minimizing less desirable components and unwanted components that can affect the quality of the sweetening properties.

### BRIEF DESCRIPTION OF THE DRAWINGS

FIG. 1 shows structures of steviol, stevioside, duloside A (DA) and related glycosides: Glc=glucose; Xyl=xylose; and Rha=rhamnose.

FIG. 2 shows a breeding diagram for obtaining high rebD Stevia plants described in more detail in the Examples.

FIG. 3 shows a plot of the percentage rebD content versus the ratio of rebA/(rebA+stevioside) based on the amount of material found in leaves as a percentage of the dry weight. Plants with the high rebD content phenotype are indicated by diamonds and plants with the wild rebD phenotype content are indicated by squares. The vertical line at 85 on the abscissa separates the plants of high and low rebD phenotype.

FIG. 4 shows a plot of the rebE (RE)+rebN (RN)+rebM (RM) content versus the rebD content found in leaves as a percentage of the dry weight. The amount of RE+RN+RM correlates significantly with the amount of rebD in plants with a correlation coefficient R²=0.86. Plants with a high rebD phenotype have an RE+RN+RM content greater than 0.4%.

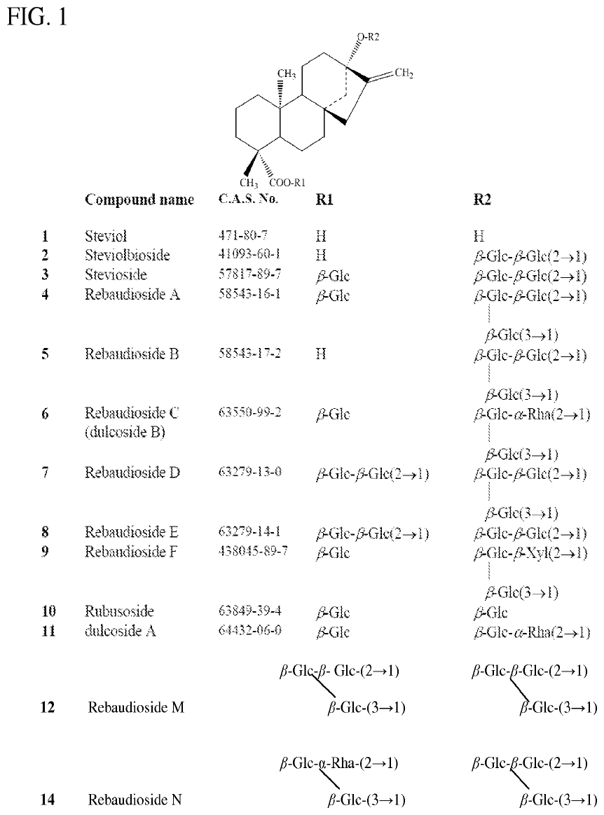

### DETAILED DESCRIPTION

### 1.0 Definitions

Rebaudiosides A, B, C, D, E, F, M, and N are denoted rebA, rebB, rebC, rebD, rebE (or RE), rebF, rebM (or RM), and rebN respectively.

As used herein high rebD plants are those with a rebD content greater than 0.5% based upon the weight of rebD in dried Stevia leaves.

As used herein high rebM plants are those with a rebM content greater than 0.1% based upon the weight of rebM in dried Stevia leaves.

As used herein high rebN plants are those with a rebN content greater than 0.1% based upon the weight of rebN in dried Stevia leaves.

It is noted that a high rebD plant may also be a high rebM and/or a high rebN plant.

A cultivar is a plant or grouping of plants produced in cultivation and selected for desirable characteristics that can be maintained by propagation. Members of a particular cultivar are not necessarily genetically identical.

A plant variety means a plant grouping within a single botanical taxon of the lowest known rank which is distinct if it can be distinguished from any other variety in that taxon of lowest rank. As used herein a variety includes identical clonal isolates propagated vegetatively.

Seed as used herein is understood to refer in the singular and/or plural to the grains or ripened ovules of Stevia plants.

As used herein “polycross” is a cross between three or more parental varieties used in selective plant breeding involving identifying plants with one or more desirable phenotypes that, upon crossbreeding with other plants of the same species, yield the most productive plants in terms of one or more desirable phenotypes. Progeny from the cross are generally propagated vegetatively and may be used to develop new varieties by subsequent cross breeding.

Weights of glycosides given throughout this disclosure refer to the percent weight of the indicated component as a percentage of the weight of the dried tissue corrected for moisture content. Weights are corrected for moisture content by drying a sample (typically 2 grams) for 3 hours at 105° C. and counting any loss of weight as due to moisture. The dried tissue typically has about 6-8% moisture.

### 2.0 Generation of Plants with Increased rebD Content

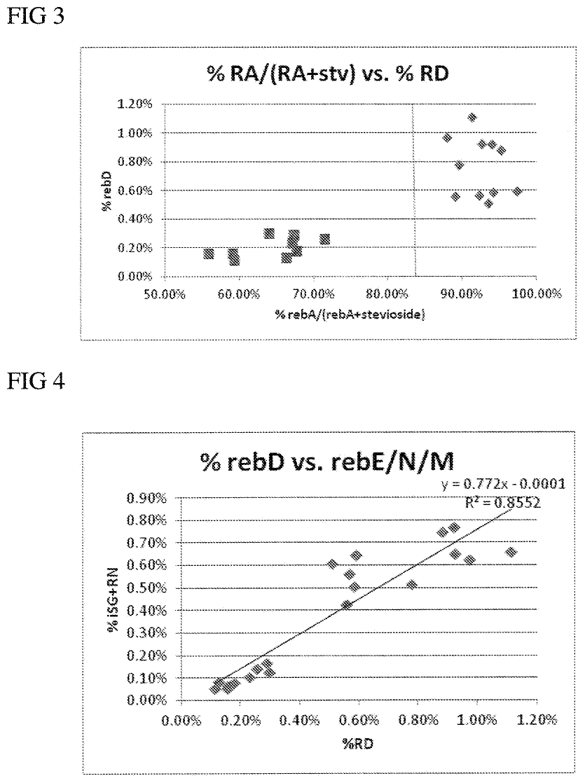

Plants with high rebD content can be seen to form a distinct population when the content of rebD and rebA/(rebA+stevioside) are plotted as shown in FIG. 3. Such plants are desirable for obtaining not only purified rebD and other rebaudiosides, but also compositions containing rebD and other glycosides including but not limited to rebA, rebE, rebM, and rebN. Such compositions are useful in the food industry as low calorie sweeteners. The compositions advantageously may contain only limited amounts of components that are bitter, or that have undesirable tastes/aftertastes to some individuals, such as stevioside.

Generation of Stevia plants with an increased rebD content and with the other desirable characteristics described herein can be accomplished by growing from the seeds transferred to the American Type Culture Collection (ATCC), 10801 University Boulevard, Manassas, Va. 20110 USA, on Mar. 15, 2013, including seeds of: SCO419×SCO435, deposit designation ______; and SCO422×SCO312, deposit designation ______. The transfer of seeds to the ATCC occurring on Mar. 15, 2013, via the Federal Express shipping service (Fed Ex) package number 3205839/INET3370 and tracking number 7992 9275 2291. A portion of the seed was transferred from ATCC in to the NCIMB, Ltd., Bucksburn, Aberdeen, Scotland, AB21 9YA where the deposits were assigned designation NCIMB 42226 Stevia rabaudiana SCO419×SCO435 Tent 1, and deposit designation NCIMB 42227 Stevia rabaudiana SCO422×SCO312 Tent 2.

It is also possible to generate varieties and lines of Stevia using the deposited lines by either conventional cross breeding techniques or molecular techniques to transfer one or more genetic elements (genes, promoters, protein coding sequences, and the like) to other Stevia plants. Alternatively, it is possible to generate Stevia plants with a high rebD content through either classical selection and cross breeding alone, or in combination with chemical or radiation induced mutation.

Described herein are high rebD Stevia plants and cultivars, having a rebD content that is greater than or equal to about 0.5%, 0.55%. 0.6%, 0.65%, 0.7%, 0.75%, 0.8%, 0.85%. 0.9%, 0.95%, 1.0%, 1.05%, or 1.1%, or a rebD content from about 0.5% to about 1%, from about 1.0% to about 1.5%, or from about 1.5% to about 2.0%. Such plants and cultivars may have a maximum rebD content of about less than 2%, 2.25%, 2.5%, 2.75%, or 3%.

In one embodiment, high rebD plants are F1, F2, F3, or subsequent progeny of high rebD Stevia plants selected from crosses of wild type plants, wherein the high rebD plants may additionally have rebA/(rebA+stevioside) ratios greater than about 0.65, 0.70, 0.75, 0.80, 0.85, 0.90, 0.92, 0.93, 0.94, or 0.95, such plants may also have a stevioside content is less than about 0.65, 0.70, 0.75, 0.80, 0.85, 0.90, 0.92, 0.93, 0.94, or 0.95. Such plants may have a maximum rebD content of about less than 2%, 2.25%, 2.5%, 2.75%, or 3%. Alternatively, the plants may have a rebD content from about 0.5% to about 1%, from about 1.0% to about 1.5%, or from about 1.5% to about 2.0%.

In another embodiment, high rebD plants are the first generation or subsequent progeny of high rebD Stevia plants whose seeds were subjected to chemical or radiation mutagenesis, wherein the high rebD plants may additionally have rebA/(rebA+stevioside) ratios greater than about 0.65, 0.70, 0.75, 0.80, 0.85, 0.90, 0.92, 0.93, 0.94, or 0.95; such plants may also have a stevioside content is less than about 0.65, 0.70, 0.75, 0.80, 0.85, 0.90, 0.92, 0.93, 0.94, or 0.95. Such plants also may have a maximum rebD content of about less than 2%, 2.25%, 2.5%, 2.75%, or 3%. Alternatively, the plants may have a rebD content from about 0.5% to about 1%, from about 1.0% to about 1.5%, or from about 1.5% to about 2.0%.

The high rebD Stevia plants described herein may have a rebA content that is greater than about 4.0%, 4.2%, 4.4%, 4.5%, 5.0%, 5.25%, or 5.5%, and which may be less than about 15%, 14%, 13%, 12%, 11.5%, 11%, 10%, 9%, 8%, 7.5%, 7.0%, 6.5%, 6.0% by weight of dried plant tissue. Stevia plants may also have a rebA content that is greater than about 4.0%, 4.2%, 4.4%, 4.5%, 5.0%, 5.25%, or 5.5% and which may be less than about 5.5%, 5.25%, 5.0%, 4.75%, or 4.6% by weight of dried plant tissue, provided the upper limit for rebA is not less than the lower limit for rebA.

In addition to the rebD and rebA content, the high rebD plants described herein may have a stevioside content that is less than about 0.65%, 0.70%, 0.75%, 0.80%, 0.85%, 0.90%, 0.92%, 0.93%, 0.94%, 0.95%, or 1.29% by weight of dried plant tissue. In one embodiment the stevioside content of high rebD plants is greater than about 0.3%, 0.35%, or 0.4% but less than about 0.65%, 0.70%, 0.75%, 0.80%, 0.85%, 0.90%, 0.92%, 0.93%, 0.94%, 0.95%, or 1.29% by weight.

The high rebD Stevia plants described herein may have a rebM content that is greater than about 0.1%, 0.2%, 0.25%, 0.3%, 0.4%, 0.5%, 0.6%, or 0.64% on dried plant tissue.

In addition to the rebD and rebM content, the high rebD plants described herein may have a rebN content that is greater than about 0.1%, 0.15%, 0.2%, 0.27%, 0.3%, 0.35%, 0.4%, 0.45%, or 0.47% on dried plant tissue.

Described herein are high rebM Stevia plants and cultivars, having a rebM content that is greater than or equal to about 0.1%, 0.2%, 0.25%, 0.26%, 0.27%, 0.28%, 0.29%, 0.30%, 0.31%, 0.4%, 0.5%, 0.6%, or 0.64%, or a rebM content from about 0.1% to about 1%, from about 0.1% to about 0.5%, or from about 0.1% to about 0.7%. Such plants and cultivars may have a maximum rebM content of about less than 0.5%, 0.7%, 1% or 2%.

Described herein are high rebN Stevia plants and cultivars, having a rebN content that is greater than or equal to about 0.1%, 0.15%, 0.2%, 0.25%, 0.26%, 0.27%, 0.28%, 0.30%, 0.35%, 0.4%, 0.45%, or 0.47%, or a rebN content from about 0.1% to about 1%, or from about 0.1% to about 0.5%. Such plants and cultivars may have a maximum rebN content of about less than 0.5%, 1% or 2%.

Another aspect of the present disclosure is directed to tissues and seeds from one or more high rebD Stevia plants or Stevia cultivars described herein. More specifically, one embodiment of this disclosure encompasses seeds of high rebD Stevia plant(s) or a Stevia cultivar. In another embodiment the disclosure is directed to a tissue of a high rebD Stevia plant or Stevia cultivars, including but not limited to leaves, stems, roots, shoots or cells. Such tissues may be in any form, including isolated, harvested, dried, powdered, lyophilized, chemically disrupted, mechanically disrupted, or physically disrupted.

As the Stevia plants described herein have a variety of uses in the food industry, it will be understood that the disclosure encompasses a composition for sweetening a food or beverage comprising any form of tissue derived from a high rebD plant. In one embodiment, the composition comprises isolated, harvested, dried, powdered, lyophilized, chemically disrupted, mechanically disrupted, or physically disrupted tissues of a high rebD plant described herein. In one such embodiment the composition comprises tissues from a high rebD plant that contained less than 1.5%, 1%, 0.9% 0.8%, 0.7%, 0.6% or 0.5% of stevioside, and which optionally has a rebA/(rebA+stevioside) ratio greater than 0.80, 0.85, 0.87 or 0.90.

Another aspect of this disclosure is directed to an extract of high rebD plant described herein. Such extracts comprise sufficient DNA and/or RNA to identify the extract as being prepared from a high rebD plant. In addition to rebD and DNA and/or RNA sufficient to identify the extract, the extract may also comprise any one or more of rebA, rebB, rebC, rebE, rebF, rebM and/or rebN. DNA and/or RNA sufficient to identify the extract as being prepared from a high rebD plant described herein will be understood to mean DNA or RNA arising from a high rebD plant or amplified nucleic acids arising from high rebD plant DNA and/or RNA present in the extract. Amplification of nucleic acids present in the sample may be accomplished by any techniques known in the art including polymerase chain reaction (PCR) and Ligase chain reaction (LCR). The DNA and/or RNA will be understood to identify the source of the extract as being a high rebD plant if analysis by molecular techniques including, but not limited to, sequencing, RFLP mapping, and the like show it to have arisen from a high rebD plant. In one embodiment, the test would establish that the extracts were derived from high rebD Stevia plants with greater than 95%, 97%, 98% or 99% confidence limits.

### 3.0 Certain Embodiments

1. One or more Stevia plants, or a Stevia cultivar wherein the leaves of said one or more plants or said cultivar (e.g., leaves harvested at 3-4 months post germination) comprise greater than about 0.5% rebD by weight of dry Stevia leaves.
2. The one or more Stevia plants or the Stevia cultivar of embodiment 1, wherein said one or more plants or said cultivar has a rebD content that is greater than or equal to about 0.5%, 0.55%. 0.6%, 0.65%, 0.7%, 0.75%, 0.8%, 0.85%. 0.9%, 0.95%, 1%, 1.05%, or 1.1% by dry weight, or a rebD content from about 0.5% to about 1%, from about 1.0% to about 1.5%, or from about 1.5% to about 2.0% by dry weight.
3. One or more Stevia plants according to any of embodiments 1-2, wherein said one or more plants have a rebA content that is less than about 15%, 14%, 13%, 12%, 11.5%, 11%, 10%, 9%, 8%, 7%, 6.5%, 6%, 5.5%, 5.25%, 5%, 4.75%, or 4.6% by dry weight.
4. One or more Stevia plants according to any of embodiments 1-3, wherein said rebA content is greater than about 4%, 4.2%, 4.4%, 4.5%, 5%, 5.25%, or 5.5% by dry weight.
5. One or more Stevia plants according to any of embodiment 1-4, wherein said stevioside content is less than about 0.65, 0.70, 0.75, 0.80, 0.85, 0.90, 0.92, 0.93, 0.94, or 1.29 percent by dry weight.
6. One or more Stevia plants according to any of embodiments 1-5, wherein said stevioside content is greater than about 0.3, 0.35, or 0.4 percent by dry weight.
7. One or more Stevia plants according to any of embodiments 1-6, wherein: the rebA/(rebA+stevioside) ratio is greater than about 0.65, 0.70, 0.75, 0.80, 0.85, 0.90, 0.92, 0.93, 0.94, or 0.95, and said ratio is less than 1.
8. One or more Stevia plants according to any of embodiments 1-7, wherein the rebA/(rebA+stevioside) ratio is greater than about 0.85, 0.90, 0.92, 0.93, 0.94, or 0.95, and said ratio is less than 1.
9. One or more Stevia plants according to any of embodiments 7-8, wherein the sum of (RebE+RebM+RebN) is greater than 0.4%, 0.5%, or 0.6% by dry weight.
10. Seed from one or more Stevia plants of any of embodiments 1-9.
11. Tissue from a plant of embodiments 1-10.
12. Leaves, stems, shoots or cells of a plant of embodiments 1-9.
13. A composition comprising one or more of a seed, tissue, leaf, stem, or cell of a plant according to any of embodiments 1-9, wherein said seed, tissue, leaf, stem, or cell is harvested, dried, powdered, lyophilized, chemically disrupted, mechanically disrupted, or physically disrupted.
14. A composition for sweetening a food or beverage comprising a composition according to embodiment 13.
15. The composition according to embodiment 13 or embodiment 14, wherein said composition comprises less than 1.5% stevioside.
16. A process for preparing a composition comprising rebD, and optionally comprising rebA, comprising preparing a composition according to embodiment 13.
17. An extract comprising rebD, and sufficient genetic material to identify it as being prepared from a plant according to any of embodiments 1-9, said extract optionally comprising any one or more of rebA, rebC, rebF.
18. A sweetener composition comprising an extract according to embodiment 17.
19. A cultivar comprising one or more high rebD plants of embodiment 1-9
20. One or more Stevia plants according to any of embodiments 1-2, wherein the content of RebM is greater than 0.1%, 0.2%, or 0.3% by dry weight.
21. One or more Stevia plants according to any of embodiments 1-2, wherein the content of RebN is greater than 0.1%, 0.2%, or 0.3% by dry weight.
22. An extract comprising rebD, and sufficient genetic material to identify it as being prepared from a plant according to any of embodiments 1-9, said extract optionally comprising any one or more of rebE, rebM, rebN.
23. A sweetener composition comprising an extract according to embodiment 22.
24. A Stevia plant or a Stevia cultivar wherein a leaf of said plant or said cultivar comprises greater than about greater than about 0.1%, 0.15%, 0.2%, 0.25%, 0.26%, 0.27%, 0.28%, 0.29%, 0.30%, or 0.31%, rebM by weight of dry Stevia tissue (e.g., leaves).
25. A Stevia plant or a Stevia cultivar wherein a leaf of said plant or said cultivar comprises greater than about 0.1%, 0.2%, 0.25%, 0.26%, 0.27%, 0.28%, 0.29%, 0.30%, 0.31%, 0.4%, 0.5%, 0.6%, or 0.64% rebM by weight of dry Stevia tissue (e.g., leaves).
26. A Stevia plant or a Stevia cultivar wherein a leaf of said plant or said cultivar comprises greater than about 0.1%, 0.15%, 0.2%, or 0.25%, 0.26%, 0.27%, or 0.28% rebN by weight of dry Stevia tissue (e.g., leaves).
27. A Stevia plant or a Stevia cultivar wherein a leaf of said plant or said cultivar comprises greater than about 0.1%, 0.15%, 0.2%, 0.25%, 0.26%, 0.27%, 0.28%, 0.3%, 0.35%, 0.4%, 0.45%, or 0.47% rebN by weight of dry Stevia tissue (e.g., leaves).
28. A Stevia plant or a Stevia cultivar wherein a leaf of said plant or said cultivar comprises greater than about 0.25%, 0.26%, 0.27%, 0.28%, 0.29%, 0.30%, 0.31% rebM and/or greater than about 0.22%, 0.25%, 0.26%, 0.27%, 0.28 rebN by weight of dry Stevia tissue (e.g., leaves).
29 A Stevia plant or a Stevia cultivar wherein a leaf of said plant or said cultivar comprises greater than about 0.25%, 0.26%, 0.27%, 0.28%, 0.29%, 0.30%, 0.31%, 0.4%, 0.5%, 0.6%, or 0.64% rebM, and/or greater than about 0.25%, 0.26%, 0.27%, 0.28%, 0.3%, 0.35%, 0.4%, 0.45%, or 0.47% rebN by weight of dry Stevia tissue (e.g., leaves).
30. A Stevia plant or a Stevia cultivar wherein a leaf of said plant or said cultivar comprises greater than about 0.4%, 0.5%, 0.6%, or 0.64% rebM, and/or greater than about 0.35%, 0.4%, 0.45%, or 0.47% rebN by weight of dry Stevia tissue (e.g., leaves).
31. A Stevia plants, or a Stevia cultivar according to any of embodiments 24-30, wherein said plants or cultivar comprise greater than about 0.87% reb D or greater than about 0.9% rebD by weight of said dry Stevia tissue.
32. A Stevia plants, or a Stevia cultivar according to embodiment 31, wherein said plants or cultivar comprises greater than about 1.0%, 1.1%, 1.2% 1.3%, 1.4%, 1.5%, or 1.6% reb D by weight of said dry Stevia tissue.
33. One or more Stevia plants according to any of embodiments 24-32, wherein said one or more Stevia plants are progeny of Stevia plants grown from one or more seed deposits selected from: ATCC deposit designation ______; ATCC deposit designation ______; NCIMB deposit designation NCIMB 42226 Stevia rabaudiana SCO419×SCO435 Tent 1; or NCIMB deposit designation NCIMB 42227 Stevia rabaudiana SCO422×SCO312 Tent 2.
34. One or more Stevia plants grown from one or more Stevia seed deposits selected from: ATCC deposit designation ______; ATCC deposit designation ______; NCIMB deposit designation NCIMB 42226 Stevia rabaudiana SCO419×SCO435 Tent 1; or NCIMB deposit designation NCIMB 42227 Stevia rabaudiana SCO422×SCO312 Tent 2.
35. A part (e.g., a leaf, stem, root, cotyledon, seed, or cell) of a Stevia plant according to any of embodiments 24-34.
36. A composition produced from a Stevia plant, or a part thereof, the composition comprising sufficient genetic material to identify it as being prepared from said Stevia plant, or a part thereof, according to any of embodiments 24-35, said composition (e.g., chemical or physical extract) optionally comprising any one, two, three, four or more of rebA, rebB, rebC, rebD, rebE, rebF, dulcoside A (DA), steviolbioside, or rubusoside.
37. A method of preparing a sweetener by extracting one or more glycosides (e.g., reb A, reb B, rebC, rebD, rebE, rebF) from a plant or part thereof according to any of embodiments 24-35.

### 4.0 Examples

### Example 1

Stevia germplasm collected from China and Argentina (AR) was classified into three genetic groups based on rebA and rebD content of high, medium and low. Four lines from each group were selected and kept in separate tents (tent1: high rebA and rebD plants, tent2: medium rebA and rebD plants and tent3: low rebA and rebD plants; see the Table 1). Seed was produced using common houseflies (Musca domestica) as pollinators in each of the tents housed in a greenhouse.

TABLE 1
The glycoside profile* of the parental clones in tents 1, 2, and 3

| | | | | | | | | Rubusoside | | Steviol | SG | 100x RA/ | Tent |
|---|---|---|---|---|---|---|---|---|---|---|---|---|---|
| | SOURCE | reb D % | reb A % | STV % | reb F % | reb C % | DA % | % | RebB | Bioside % | (J9) % | (RA + STV) | # |
| SCO 73 | China | 0.28 | 8.73 | 4.07 | 0.89 | 0.76 | 0.08 | 0.07 | 0.00 | 0.00 | 14.88 | 68.18 | 1 |
| c.7.6.11 2 | China | 0.47 | 12.21 | 6.75 | 1.07 | 1.17 | 0.08 | 0.08 | 0.00 | 0.00 | 21.83 | 64.41 | 1 |
| c.7.6.11 19 | AR | 0.27 | 10.11 | 4.61 | 0.83 | 1.00 | 0.04 | 0.06 | 0.00 | 0.00 | 16.92 | 68.70 | 1 |
| C.7.6.11 3 | AR | 0.21 | 9.66 | 5.09 | 0.72 | 1.08 | 0.08 | 0.00 | 0.00 | 0.00 | 16.83 | 65.50 | 1 |
| SCO 90 | China | 0.12 | 4.30 | 8.83 | 0.56 | 0.62 | 0.21 | 0.29 | 0.45 | 0.04 | 15.43 | 32.76 | 2 |
| c.7.6.11 48 | China | 0.19 | 5.81 | 6.81 | 0.72 | 0.86 | 0.13 | 0.05 | 0.00 | 0.00 | 14.57 | 46.03 | 2 |
| c.7.6.11 43 | AR | 0.20 | 5.43 | 9.47 | 0.60 | 0.79 | 0.24 | 0.12 | 0.00 | 0.00 | 16.85 | 36.43 | 2 |
| c.7.6.11 30 | AR | 0.19 | 6.32 | 6.57 | 0.54 | 0.82 | 0.10 | 0.05 | 0.00 | 0.00 | 14.58 | 49.01 | 2 |
| SCO 71 | China | 0.11 | 1.00 | 7.30 | 1.16 | 0.33 | 0.20 | 0.03 | 0.00 | 0.00 | 10.13 | 12.05 | 3 |
| c.7.6.11 14 | AR | o.10 | 1.73 | 6.37 | 0.67 | 0.41 | 0.20 | 0.00 | 0.00 | 0.00 | 9.47 | 21.37 | 3 |
| SCO 179 | China | 0.11 | 3.99 | 7.70 | 0.64 | 0.54 | 0.20 | 0.45 | 0.00 | 0.00 | 13.63 | 34.12 | 3 |
| SCO 170 | China | 0.11 | 3.72 | 5.62 | 0.60 | 0.45 | 0.14 | 0.29 | 0.00 | 0.00 | 10.95 | 39.85 | 3 |

*Glycoside profile percent (%) weight is given as the % of tissue weight corrected for moisture content. See the protocol use for drying leaves of Stevia plants in Example 2.

Seed was harvested from each tent. A portion of the harvested seed was subjected to gamma radiation at 10, 25 or 50 kilo rads using a cobalt-60 (⁶⁰Co) source (Department of Environmental and Radiological Health Sciences, College of Veterinary Medicine and Biomedical Sciences, Colorado State University). Table 2 summarizes the amount of seed harvested from each tent, the portions of seed subjected to each irradiation treatment, and the amount kept as control non-irradiated seeds.

TABLE 2
Seed harvested from tents 1-3, and the amounts subjected
to each irradiation treatment and employed as a control

| | total seed harvested | seeds for | seeds for irradation (g) | | |
|---|---|---|---|---|---|
| | in grams (g)* | control (g) | 10 k-rad | 25 k-rad | 50 k-rad |
| tent1 | 5.6 | 0.5 | 1.5 | 1.5 | 1.5 |
| tent2 | 10.8 | 0.5 | 3 | 3 | 3 |
| tent3 | 17.8 | 0.5 | 5 | 5 | 5 |

*1000 seeds produced under greenhouse conditions have an average weight of about 0.4 grams

The irradiated (mutagenized) and control seed were sowed into soil and allowed to germinate in growth chambers held at about 24° C. with 24 hours of light. Seed began to germinate in about 3-4 days, and after about a week from planting the seedlings were moved into a greenhouse for growth. Plants grown from the irradiated seed were designated M₀ generations plants. Table 3 summarizes the number of plants generated from seed from each tent subject to different dosages of radiation.

TABLE 3
The number of plants generated from seed (seedlings) of
each tent subject to the specified radiation dosages

| | control | 10 k-rad | 25 k-rad | 50 k-rad |
|---|---|---|---|---|
| tent1 | 65 (from SCO 239 | 112 (from SCO 304 | 56 (from SCO 416 | 47 (from SCO 472 |
| | to SCO 303) | to SCO 415) | to SCO 471) | to SCO 518) |
| tent2 | 72 (from SCO 519 | 231 (from SCO 591 | 69 (from SCO 822 | 11 (from SCO 891 |
| | to SCO 590) | to SCO 821) | to SCO 890) | to SCO 901) |
| tent3 | 88 (from SCO 902 | 526 (from SCO 990 | 396 (from SCO 1516 | 142 (from SCO 1912 |
| | to SCO 989) | to SCO 1515) | to SCO 1911) | to SCO 2053) |

When the plants listed in Table 3 were three to four months old, the leaf tissues (75% from top one-third and 25% from middle one-third) were collected from each individual plant of the control and irradiated mutant plants of tent1. The leaves were dried in a dehydrator for 20-24 hours at 68° C. The dried leaf samples were analyzed for glycoside profile by High Pressure Thin Layer Chromatography (HPTLC) and/or High Performance Liquid Chromatography (HPLC, sometimes referred to as High Pressure Liquid Chromatography).

High rebD plants from tent1 were analyzed by HPLC and the average glycoside composition among non-radiated control, 10 krad (kilorads), 25 had, and 50 krad shown in Table 4 suggested that 25 krad on average produced plants with higher rebD content and higher rebA/(rebA+stevioside) ratio. See Table 4.

TABLE 4
The average glycoside composition given as % dry weight of leaves from all plants under each
irradiation treatment and control and the ratio rebA/(rebA + STV) for plants from tent 1.

| | | | | | | | Rubus- | | Steviol | | 100 × rebA |
|---|---|---|---|---|---|---|---|---|---|---|---|
| | rebD | rebA | STV | rebF | rebC | DA | oside | rebB | Bioside | SG | (rebA + STV) |
| Control | 0.26 | 5.86 | 2.58 | 0.54 | 0.51 | 0.05 | 0.03 | 0.02 | 0.01 | 9.87 | 69.46 |
| 10k-rad | 0.25 | 5.21 | 2.84 | 0.57 | 0.48 | 0.06 | 0.03 | 0.02 | 0.02 | 9.48 | 64.72 |
| 25k-rad | 0.36 | 4.68 | 1.69 | 0.49 | 0.37 | 0.04 | 0.03 | 0.02 | 0.01 | 7.69 | 73.54 |
| 50k-rad | 0.25 | 7.09 | 3.18 | 0.5 | 0.64 | 0.06 | 0.04 | 0.03 | 0.02 | 11.83 | 69.03 |

Chromatography showed several plants from tent 1 arising out of the irradiated group produced rebD at levels higher than control plants and their parents. The mutant SCO312 had 1.11% rebD at the early flowering stage, which was about eight months old. See Table 6. More than 350 plants out of a total of 1535 plants from tents 2 and 3 were analyzed; however, no plants were found to have a glycoside profile with a significant change, particularly in rebD content, compared with control and/or parental plants. See Example 3.

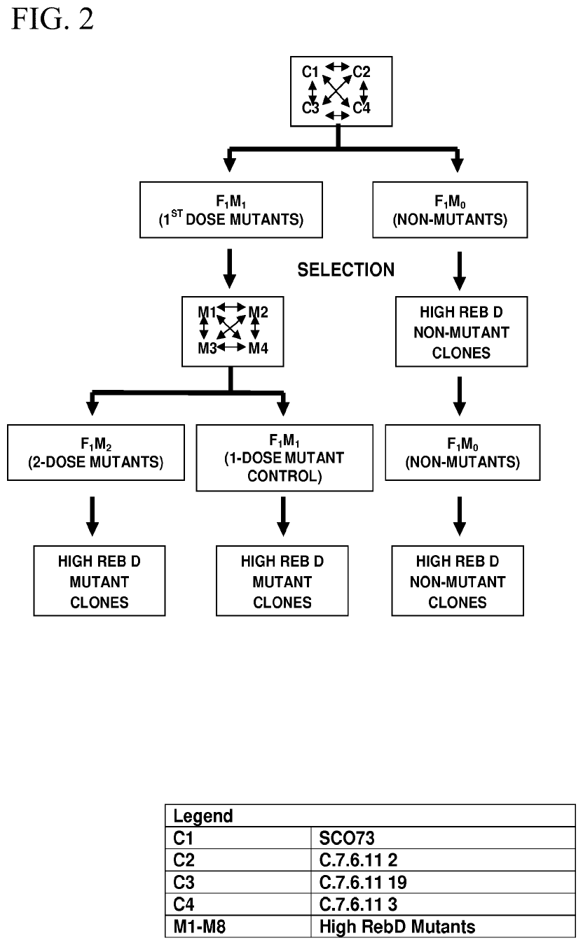

The leaf tissue collection and analysis were continuously carried out in the following several months until the control and plants arising from irradiated seed started flowering. The control and high rebD, rebA and rebA/(rebA+STV) plants were selected. Two bi-parental crosses with high reb D parents selected in M0 population (SCO 422×SCO 312 and SCO 435×SCO 419) and one bi-parental cross with high rebD parents selected in F1 population (SCO 273×SCO 260) were made (FIG. 2). Seed from crosses SCO 422×SCO 312 and SCO 435×SCO 419 was divided into two pools and one half given a dose of 20k-rads of gamma radiation as described above. Seed from the second round of gamma irradiation, designated as “Ml” and seeds of non-irradiated “Ml” and “F1” plants were planted as described above to generate the next generation of plants for selection.

The results show that mutagenesis, and specifically gamma radiation induced mutagenesis, can generate plants with higher rebD content and higher rebA/(rebA+stevioside) ratio than plants by traditional breeding techniques (see Table 6).

### Example 2 Analysis of Glycosides in Stevia Plants by HPLC Analysis

### Example 2 (Part A) Leaf Extraction

Leaves harvested from Stevia plants are dried at about 68° C. for 24 hours and stored intact until they are to be analyzed. Leaves are ground to dry leaf powder. Ten ml of water is added to 0.1 g of leaf powder in a capped 15 mL tube. After mixing to wet the leaf powder, the sample is placed in a 50° C. water bath for one hour where it is mixed every 15 minutes. The sample is filtered through a 0.45 nm nylon filter and placed into a liquid chromatography vial.

### Example 2 (Part B) Chromatography of Glycoside Samples

Filtered samples from 2A, above, are applied to a High Performance Liquid Chromatography (HPLC) system with heating (Waters 2695, or equivalent) equipped with a UV-Vis detector (Waters 2487 dual wavelength, or equivalent). For the analysis the HPLC is outfitted with a 250 mm×4.6 mm, 5 μm C18 column (Capcell pak MGII, Shiseido Co. Ltd. or equivalent) and a guard column (Phenomenex, KJO-4282 with a Phenomenex, C18 4×2.0 mm, P/N AJO-4286 insert or equivalent). The samples are prepared in water and analyzed by a gradient elution method shown in the table below.

Solvent A: 0.01M phosphate buffer pH 2.6; solvent B: acetonitrile. Flow rate: 1 ml/min: column temperature 55° C.

| Time (min) | Solvent A | Solvent B |
|---|---|---|
| 0 | 80% | 20% |
| 5.5 | 80% | 20% |
| 8 | 70% | 30% |
| 11 | 70% | 30% |
| 14.5 | 65% | 35% |
| 23 | 65% | 35% |
| 26.5 | 20% | 80% |
| 29 | 20% | 80% |
| 31.5 | 80% | 20% |

Detection of glycosides is conducted at 210 nm. Data collection and handling software (e.g., Empower, Waters, Corp. or equivalent) are employed to determine glycoside content of samples using standards of known glycosides for calibration.

To determine the amount of glycosides in plant tissues, the weight of leaves or other tissue used in the extraction are corrected for moisture content by drying a weighed sample of the leaf powder at 105° C. for 3 hours and determining the weight loss which is attributed to moisture.

### Example 3 Glycoside Analysis of Stevia Plants Derived by Traditional Breeding and Gamma Mutagensis

The glycoside content of parent plants (see Table 5 where each parental isolate was assessed at two different times) and selected progeny developed by the breeding scheme in Example 1 is analyzed by HPLC using the method outlined in Example 2. Plants are considered to be high rebD plants if they contain greater than 0.5% rebD based on the weight of the glycoside in dry leaves corrected for moisture. A subse of high rebD plants contains greater than 0.5% rebD and has a rebA/(rebA+stevioside) ratio greater than 0.8 (or expressed as a percentage 80%). In addition to high rebD, the plants described below may contain high levels of higher glycosides, such as rebN. Such plants many contain greater than 0.5% rebD and greater than 0.4% combined rebE+rebN+rebM, and a higher total glycoside content.

TABLE 5
The glycoside profile of leaves from parental Stevia plants that do not contain a high
rebD content and do not have a rebA/(rebA + stevioside) ratio greater than 0.8 or high rebD
isomer content.

| | | | | | | | Rubus- | | Steviol | |
|---|---|---|---|---|---|---|---|---|---|---|
| | rebD | rebA | STV | rebF | rebC | DA | oside | rebB | Bioside | SG |
| SCO 7.6.11.19 jan | 0.16% | 6.69% | 4.64% | 0.56% | 0.63% | 0.05% | 0.07% | 0.02% | 0.01% | 12.85 |
| SCO 7.6.11.19 orig | 0.26% | 9.76% | 3.91% | 0.55% | 0.88% | 0.04% | 0.03% | 0.04% | 0.01% | 15.47 |
| SCO 7.6.11.2 feb | 0.11% | 5.77% | 3.96% | 0.80% | 0.58% | 0.06% | 0.08% | 0.02% | 0.01% | 11.39 |
| SCO 7.6.11.2-01 | 0.30% | 10.21% | 5.76% | 0.68% | 0.99% | 0.05% | 0.05% | 0.04% | 0.02% | 18.08 |
| SCO 7.6.11.2-02 | 0.29% | 9.82% | 4.78% | 0.55% | 0.94% | 0.03% | 0.03% | 0.03% | 0.01% | 16.49 |
| SCO 7.6.11.3 jan | 0.16% | 7.16% | 5.67% | 0.62% | 0.81% | 0.10% | 0.03% | 0.02% | 0.02% | 14.60 |
| SCO 7.6.11.3 orig | 0.23% | 8.66% | 4.25% | 0.52% | 0.87% | 0.07% | 0.03% | 0.03% | 0.02% | 14.67 |
| SCO73 jan | 0.18% | 7.34% | 3.52% | 0.82% | 0.62% | 0.08% | 0.04% | 0.01% | 0.01% | 12.62 |
| SCO73 orig | 0.13% | 6.40% | 3.27% | 0.65% | 0.59% | 0.07% | 0.02% | 0.01% | 0.01% | 11.14 |

| | rebD | rebA/(rebA + stv) | rebE | rebN | rebM | rebE + rebM + rebN |
|---|---|---|---|---|---|---|
| SCO 7.6.11.19 jan | 0.16% | 59.04% | 0.01% | 0.02% | 0.03% | 0.06% |
| SCO 7.6.11.19 | 0.26% | 71.42% | 0.03% | 0.04% | 0.06% | 0.13% |
| SCO 7.6.11.2 fe b | 0.11% | 59.26% | 0.02% | 0.01% | 0.02% | 0.05% |
| SCO 7.6.11.2-01 | 0.30% | 63.92% | 0.01% | 0.09% | 0.02% | 0.13% |
| SCO 7.6.11.2-02 | 0.29% | 67.26% | 0.04% | 0.05% | 0.07% | 0.16% |
| SCO 7.6.11.3 j an | 0.16% | 55.80% | 0.01% | 0.02% | 0.01% | 0.05% |
| SCO 7.6.11.3 orig | 0.23% | 67.08% | 0.02% | 0.05% | 0.04% | 0.10% |
| SCO73 jan | 0.18% | 67.60% | 0.02% | 0.03% | 0.03% | 0.07% |
| SCO73 orig | 0.13% | 66.22% | 0.02% | 0.03% | 0.03% | 0.08% |

SG is the sum of rebD, rebA, stevioside (STV), rebF, rebC, dulcoside A(DA), rubusoside, and rebB.

High rebD plants from this study, including both progeny arising from conventional breeding (SCO 260-300) and progeny derived from gamma radiation-induced mutation breeding (SCO312-512) also show a high rebA ratio (rebA/(rebA+stevioside)) and a high content of rebE, rebM, and rebN. See Table 6.

TABLE 6
The glycoside profile of leaves from selected Stevia plants bred to contain a high
rebD content.

| | | | | | | | | | Steviol | |
|---|---|---|---|---|---|---|---|---|---|---|
| Sample | rebD | rebA | STV | rebF | rebC | DA | Rubusoside | rebB | Bioside | SG |
| SCO260 | 0.78% | 11.07% | 1.29% | 0.39% | 0.84% | 0.04% | 0.01% | 0.07% | 0.02% | 14.51% |
| SCO273 | 0.92% | 8.03% | 0.64% | 0.65% | 0.49% | 0.07% | 0.02% | 0.06% | 0.01% | 10.88% |
| SCO284 | 0.57% | 6.69% | 0.56% | 0.62% | 0.44% | 0.09% | 0.03% | 0.05% | 0.01% | 9.06% |
| SCO296 | 0.59% | 5.41% | 0.15% | 0.22% | 0.33% | 0.05% | 0.01% | 0.02% | 0.00% | 6.78% |
| SCO300 | 0.56% | 4.15% | 0.51% | 0.57% | 0.31% | 0.04% | 0.01% | 0.02% | 0.00% | 6.18% |
| SCO312 | 1.11% | 8.42% | 0.80% | 0.60% | 0.63% | 0.05% | 0.02% | 0.05% | 0.01% | 11.70% |
| SCO419 | 0.97% | 6.17% | 0.85% | 0.52% | 0.39% | 0.04% | 0.02% | 0.04% | 0.01% | 9.01% |
| SCO422 | 0.88% | 7.99% | 0.40% | 0.52% | 0.56% | 0.06% | 0.02% | 0.04% | 0.00% | 10.47% |
| SCO427 | 0.58% | 5.14% | 0.32% | 0.38% | 0.28% | 0.05% | 0.02% | 0.02% | 0.00% | 6.80% |
| SCO435 | 0.92% | 6.75% | 0.43% | 0.38% | 0.43% | 0.05% | 0.01% | 0.03% | 0.00% | 9.00% |
| SCO512 | 0.51% | 7.01% | 0.49% | 0.36% | 0.48% | 0.06% | 0.03% | 0.06% | 0.00% | 9.00% |

| Sample | rebD | rebA/(rebA + stv) | rebE | rebN | reb M | rebE + rebM + rebN |
|---|---|---|---|---|---|---|
| SCO260 | 0.78% | 89.53% | 0.10% | 0.18% | 0.21% | 0.49% |
| SCO273 | 0.92% | 92.65% | 0.15% | 0.20% | 0.27% | 0.61% |
| SCO284 | 0.57% | 92.30% | 0.10% | 0.17% | 0.26% | 0.52% |
| SCO296 | 0.59% | 97.29% | 0.14% | 0.14% | 0.31% | 0.60% |
| SCO300 | 0.56% | 89.02% | 0.07% | 0.20% | 0.14% | 0.41% |
| SCO312 | 1.11% | 91.32% | 0.15% | 0.28% | 0.20% | 0.63% |
| SCO419 | 0.97% | 87.93% | 0.17% | 0.17% | 0.25% | 0.59% |
| SCO422 | 0.88% | 95.20% | 0.17% | 0.22% | 0.31% | 0.70% |
| SCO427 | 0.58% | 94.17% | 0.06% | 0.15% | 0.26% | 0.47% |
| SCO435 | 0.92% | 93.95% | 0.14% | 0.28% | 0.31% | 0.72% |
| SCO512 | 0.51% | 93.46% | 0.11% | 0.15% | 0.30% | 0.56% |

The graphs in FIG. 3 and FIG. 4 show the distinct separation of high rebD progeny from low rebD parent plants and the correlation between the rebD content in plants vs. rebE+rebM+rebN content in plants.

### Example 4: Glycoside Analysis of Additional Stevia Plants Derived by Subsequent Gamma Mutagenesis

Several stevia plants produced by gamma mutagenesis from Example 1 were poly-crossed to produce seeds. The seeds from the poly-crossed plants were subject a second gamma induced mutagenesis using the same method as described in Example 1, and then germinated to produce plants. The leaves from select plants subject to the second mutagenesis were dried and analyzed as described in Examples 2 and 3. The glycoside content of the leaves are listed in table 7.

TABLE 7
The glycoside profile of leaves from selected Stevia plants bred to contain a high rebD,
high rebM, and high rebN content

| Sample | | | | | | |
|---|---|---|---|---|---|---|
| Name | Reb E | Reb D | Reb N | Reb M | Reb A | Stevioside |
| 2300-02 | 0.08% | 1.00% | 0.35% | 0.49% | 6.94% | 0.82% |
| 2300-12 | 0.03% | 0.72% | 0.19% | 0.33% | 5.41% | 1.16% |
| 2300-13 | 0.10% | 1.43% | 0.35% | 0.43% | 3.24% | 0.56% |
| 2303-05 | 0.08% | 0.95% | 0.36% | 0.62% | 8.27% | 0.47% |
| 2303-32 | 0.04% | 0.79% | 0.25% | 0.43% | 10.22% | 1.09% |
| 2303-33 | 0.04% | 0.69% | 0.22% | 0.23% | 4.92% | 0.60% |
| 2303-13 | 0.08% | 1.43% | 0.38% | 0.51% | 6.59% | 0.68% |
| 2303-14 | 0.04% | 1.04% | 0.28% | 0.45% | 6.86% | 0.51% |
| 2303-26 | 0.05% | 1.05% | 0.36% | 0.49% | 7.18% | 0.65% |
| 2300-09 | 0.13% | 0.98% | 0.27% | 0.53% | 4.94% | 0.30% |
| 2303-15 | 0.05% | 1.19% | 0.26% | 0.48% | 7.13% | 0.76% |
| 2303-38 | 0.07% | 0.88% | 0.25% | 0.37% | 10.45% | 1.64% |
| 2305-01 | 0.04% | 0.86% | 0.34% | 0.58% | 8.20% | 0.79% |
| 2303-48 | 0.05% | 1.02% | 0.34% | 0.49% | 10.22% | 1.47% |
| 2303-49 | 0.04% | 0.75% | 0.21% | 0.26% | 8.78% | 1.37% |
| 2304-43 | 0.09% | 1.18% | 0.33% | 0.39% | 2.28% | 0.59% |
| 2303-02 | 0.04% | 1.13% | 0.31% | 0.55% | 6.88% | 0.47% |
| 2305-18 | 0.07% | 1.62% | 0.48% | 0.65% | 8.62% | 0.87% |
| 2303-18 | 0.03% | 1.07% | 0.26% | 0.47% | 6.56% | 0.70% |

As seen Table 7, plants have elevated rebD, rebM, and/or rebN content appear among the plants produced from seeds subject to the second round of gamma mutagenesis.

1.-19. (canceled)

20. A Stevia plant grown from NCIMB deposit designation NCIMB 42226 Stevia rabaudiana SCO419×SCO435 Tent 1 or NCIMB deposit designation NCIMB 42227 Stevia rabaudiana SCO422×SCO312 Tent 2, or progeny of such a Stevia plant, having leaves that comprise greater than 0.55% Rebaudioside D (rebD) by dry weight.

21. The Stevia plant of claim 20, wherein the leaves have a rebD content that is greater than or equal to 0.75% by dry weight.

22. The Stevia plant of claim 20, wherein the leaves have a rebD content that is greater than or equal to 1% by dry weight.

23. The Stevia plant of claim 20, wherein the leaves have a rebD content that is greater than or equal to 0.7% by dry weight and less than 3% by dry weight.

24. The Stevia plant of claim 20, wherein the leaves have a Rebaudioside A (rebA) content that is less than 15% by dry weight.

25. The Stevia plant of claim 20, wherein the leaves have a stevioside content that is less than 0.65% by dry weight.

26. The Stevia plant of claim 20, wherein the leaves have a rebA content and a stevioside content and a rebA/(rebA+stevioside) ratio greater than 0.65 and less than 1.

27. The Stevia plant of claim 20, wherein the leaves have a sum of a Rebaudioside E (rebE) content, a Rebaudioside M (rebM) content, and a Rebaudioside N (rebN) content that is greater than 0.4% by dry weight.

28. The Stevia plant of claim 20, wherein the leaves have a sum of a rebE content, a rebM content, and a rebN content that is greater than 0.6% by dry weight.

29. A Stevia extract comprising rebD and genetic material from a Stevia plant of claim 20.

30. The Stevia extract of claim 29, wherein the genetic material is DNA, RNA, or a combination thereof.

31. A process for preparing a sweetening composition comprising extracting steviol glycosides from leaf of a Stevia plant grown from NCIMB deposit designation NCIMB 42226 Stevia rabaudiana SCO419×SCO435 Tent 1 or NCIMB deposit designation NCIMB 42227 Stevia rabaudiana SCO422×SCO312 Tent 2, or progeny of such a Stevia plant, said plant having leaves that comprise greater than 0.55% rebD by dry weight.

32. A sweetening composition produced in accordance with claim 31.

33. A beverage sweetened with a sweetening composition produced in accordance with claim 31.
